(19) United States
(12) Patent Application Publication
(10) Pub. No.: US 2025/0352676 A1
(43) Pub. Date: Nov. 20, 2025

(54) N-Cyclopropyl-1-(4-(4-(Fluoro-18f)Phenyl)Pyrimidin-5-Yl)-N-Methylpiperidine- 4-Carboxamide and Uses in PET Imaging

(71) Applicant: Emory University, Atlanta, GA (US)

(72) Inventors: Huan Liang, Atlanta, GA (US); Ahmed Haider, Atlanta, GA (US); Chunyu Zhao, Atlanta, GA (US)

(21) Appl. No.: 19/103,467

(22) PCT Filed: Aug. 11, 2023
(86) PCT No.: PCT/US2023/030068

§ 371 Date: Feb. 12, 2025

Related U.S. Application Data
(60) Provisional application No. 63/397,463, filed on Aug. 12, 2022.

(51) Int. Cl. A61K 51/04; C07B 59/00; C07D 401/04; C07F 5/02; C12N 5/00

(52) U.S. Cl. CPC A61K 51/0459; C07B 59/002; C07D 401/04; C07F 5/025; C12N 5/0018; A61K 2123/00; C07B 2200/05

(57) ABSTRACT

Disclosed herein is a PET tracer compound N-cyclopropyl-1-(4-(4-(fluoro-18F) phenyl)pyrimidin-5-yl)-N-methyl piperidine-4-carboxamide or salt thereof, and uses as an imaging agent. In certain embodiments, this disclosure relates to precursor compounds such as N-cyclopropyl-N-methyl-1-(4-(4-(4,4,5,5-tetramethyl-1,3,2-dioxaborolan-2-yl) phenyl)pyramidin-5-yl)piperidine-4-carboxamide or salt thereof and kits comprising the same.

### CROSS-REFERENCE TO RELATED APPLICATIONS

This application claims the benefit of U.S. Provisional Application No. 63/397,463 filed Aug. 12, 2022. The entirety of this application is hereby incorporated by reference for all purposes.

### STATEMENT REGARDING FEDERALLY SPONSORED RESEARCH OR DEVELOPMENT

This invention was made with government support under AG070060 awarded by the National Institutes of Health. The government has certain rights in the invention.

### BACKGROUND

Brain cholesterol homeostasis orchestrates the biosynthesis, transport, metabolism, and clearance of cholesterol from the mammalian central nervous system (CNS). Exchange of plasma and brain cholesterol is precluded by the blood-brain barrier. Brain cholesterol is synthesized by astrocytes and neurons. HMG-CoA reductase catalyzes the rate-limiting step in the biosynthesis of cholesterol, and cholesterol 24-hydroxylase, also referred to as cytochrome P450 46A1 or CYP46A1, facilitates the clearance of cholesterol from the CNS by mediating the conversion of cholesterol to 24-(S)-hydroxycholesterol, also referred to as hydroxycholesterol or 24S-hydroxycholesterol, which is a metabolite that readily penetrates the blood-brain barrier.

Cholesterol turnover has physiological relevance in synaptic plasticity, learning, and memory. The concentration of cholesterol in the brain is implicated in a variety of neurodegenerative, inflammatory, and vascular brain diseases. Underlying mechanisms are not fully understood. Hydroxycholesterol has been suggested as a potential biomarker for Alzheimer's, Huntington's disease, autism, epilepsy, depression, and Parkinson's disease. Attempts to assess plasma concentrations of hydroxycholesterol as a surrogate measure for CYP46A1 activity in the brain have yielded conflicting results. An important consideration is that hydroxycholesterol is highly susceptible to metabolism in the liver. Hence, the correlation of hydroxycholesterol plasma concentrations with CYP46A1 function is confounded by downstream metabolic processes in the periphery, raising substantial concerns that a plasma biomarker does not accurately reflect brain cholesterol metabolism. Thus, there is a need to identify procedures to assess or estimate cholesterol, hydroxycholesterol, or enzymes that alter their concentrations in the brain.

Popp et al report cerebral and extracerebral cholesterol metabolism and CSF markers of Alzheimer's disease. Biochem. Pharmacol. 86, 37-42 (2013).

Shafaati et al. report levels of ApoE in cerebrospinal fluid are correlated with Tau and 24S-hydroxycholesterol in patients with cognitive disorders. Neurosci. Lett. 425, 78-82 (2007).

Papassotiropoulos et al. report 24S-hydroxycholesterol in cerebrospinal fluid is elevated in early stages of dementia. J. Psychiatr. Res. 36, 27-32 (2002).

Leoni et al. report CSF levels of 24S-hydroxycholesterol may be a biomarker for mild cognitive impairment. Neurosci. Lett. 397, 83-87 (2006).

Besga et al. report in brain cholesterol metabolism and insulin in two subgroups of patients with different CSF biomarkers but similar white matter lesions suggest different pathogenic mechanisms. Neurosci. Lett. 510, 121-126 (2012).

Koike et al. report preclinical characterization of [18F]T-008, a PET imaging radioligand for cholesterol 24-hydroxylase. European Journal of Nuclear Medicine and Molecular Imaging (2022) 49:1148-1156. See also US Pat. Pub. No. 2017/0114042.

Koike et al. report radiolabeled compounds useful as radiotracers for quantitative imaging in mammals. PCT Publication No. WO2015/190613.

References cited herein are not an admission of prior art.

### SUMMARY

This disclosure relates to a tracer compound N-cyclopropyl-1-(4-(4-(fluoro-18F)phenyl) pyrimidin-5-yl)-N-methyl piperidine-4-carboxamide or salts thereof, and uses as a PET imaging agent. In certain embodiments, this disclosure relates to precursor compounds for generating N-cyclopropyl-1-(4-(4-(fluoro-18F)phenyl)pyrimidin-5-yl)-N-methylpiperidine-4-carboxamide such as N-cyclopropyl-N-methyl-1-(4-(4-(4,4,5,5-tetramethyl-1,3,2-dioxaborolan-2-yl)phenyl) pyrimidin-5-yl)piperidine-4-carboxamide or salts thereof.

In certain embodiments, this disclosure relates to methods comprising: a) administering a composition comprising the tracer compound N-cyclopropyl-1-(4-(4-(fluoro-18F)phenyl)pyrimidin-5-yl)-N-methyl piperidine-4-carboxamide isotopically enriched with fluorine 18 to a subject; and scanning the subject for emissions from an area of the subject. In certain embodiments, the N-cyclopropyl-1-(4-(4-(fluoro-18F)phenyl)pyrimidin-5-yl)-N-methylpiperidine-4-carboxamide tracer passes the blood brain barrier and emissions are from inside the brain or skull area.

In certain embodiments, methods further comprise the step of detecting and/or measuring the emissions and creating an image indicating or highlighting the location of the compound isotopically enriched with fluorine 18 in the subject.

In certain embodiments, methods comprise the step of detecting, measuring, and/or quantifying the emission providing an emission quantity and optionally correlating the emission measurement/detection/quantity to a concentration of cholesterol 24-hydroxylase (cytochrome P450 46A1) and/or 24S-hydroxycholesterol in the tissue, e.g., brain.

In certain embodiments, methods further comprise the step of correlating a low, high, or abnormal measurement, quantity, or concentration of cholesterol 24-hydroxylase (cytochrome P450 46A1) and/or 24-hydroxycholesterol to the existence of or diagnosis of a subject at risk of a central nervous system disease or condition. In certain embodiments, the disease is Alzheimer's disease. Huntington's disease, autism, epilepsy. depression, and Parkinson's disease, mild cognitive impairment, or other cognitive disorder or neurodegenerative disease.

In certain embodiments, this disclosure relates to a PET imaging precursor compound N-cyclopropyl-N-methyl-1-(4-(4-(4,4,5,5-tetramethyl-1,3,2-dioxaborolan-2-yl)phenyl)pyrimidin-5-yl)piperidine-4-carboxamide or salts thereof.

In certain embodiments, this disclosure relates to method of making the N-cyclopropyl-1-(4-(4-(fluoro-18F)phenyl)pyrimidin-5-yl)-N-methylpiperidine-4-carboxamide tracer comprising contacting a precursor compound N-cyclopropyl-N-methyl-1-(4-(4-(4,4,5,5-tetramethyl-1,3,2-dioxaborolan-2-yl)phenyl)pyrimidin-5-yl)piperidine-4-carboxamide with an isotopically enriched fluorine 18 negative ion producing the PET imaging tracer N-cyclopropyl-1-(4-(4-(fluoro-18F)phenyl)pyrimidin-5-yl)-N-methyl piperidine-4-carboxamide.

In certain embodiments, the enriched fluorine 18 negative ion is a fluorine 18 potassium salt bound to a cryptand.

In certain embodiments, this disclosure relates to kits comprising a precursor PET imaging compound disclosed herein and potassium ion bound to a cryptand.

### BRIEF DESCRIPTION OF THE SEVERAL VIEWS OF THE DRAWINGS

FIG. 1A illustrates the PET imaging compound 18F-CHL-2205 (18F-Cholestify) with the chemical name N-cyclopropyl-1-(4-(4-(fluoro-18F) phenyl) pyrimidin-5-yl)-N-methylpiperidine-4-carboxamide, and its preparation from the boron precursor, N-cyclopropyl-N-methyl-1-(4-(4-(4,4,5,5-tetramethyl-1,3,2-dioxaborolan-2-yl)phenyl)pyrimidin-5-yl)piperidine-4-carboxamide.

FIG. 1B illustrates CYP46A1-mediated hydroxylation of cholesterol to 24S-hydroxycholesterol in the central nervous system (CNS).

FIG. 1C shows data from PET scans from an Alzheimer's disease mouse model, 3×Tg-AD, and respective control animals. Top: time-activity curves (TACs) under baseline conditions, where only the tracer is administered. Bottom: TACs under blockade conditions, where the tracer is administered together with an excess of nonradioactive CHL-2205 to diminish the number of enzymes that are available for tracer-CYP46A1 interactions.

FIGS. 2A-2F show data indicating the ability to quantitative assess cholesterol metabolism in the living human brain.

FIG. 2A shows data from representative PET images of the human brain reflecting CYP46A1-rich brain regions, averaged from 0 to 90 min after tracer injection. Caudate/putamen are CYP46A1-rich regions, and the cerebellum is a CYP46A1-poor region. Quantitative data are depicted as standardized uptake values (SUVs) from respective individual scans.

FIG. 2B shows data from the distribution of 18F-CHL-2205 in the human brain presented as SUVs from 15 to 30 min after injection (SUV15-30) for the respective individual scans. FIG. 2C shows a kinetic modeling assessment of tissue volumes of distribution (VT) for 18F-CHL-2205 in the human brain.

FIG. 2D shows data using a kinetic modeling assessment of nondisplaceable binding potentials (BPND) for 18F-CHL-2205 in the human brain.

FIG. 2E shows a correlation of PET signals averaged from 15 to 30 min after tracer injection with nondisplaceable binding potentials (BP_{ND}) in selected brain regions.

FIG. 2F shows analysis of postmortem human brain specimens by correlation of PET signals with Western blot analysis in selected brain regions.

### DETAILED DISCUSSION

Before the present disclosure is described in greater detail, it is to be understood that this disclosure is not limited to particular embodiments described, and as such may, of course, vary. An “embodiment” of this disclosure refers to an example and infers that the example is not necessarily limited to the example. It is also to be understood that the terminology used herein is for the purpose of describing particular embodiments only, and is not intended to be limiting. since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. Prior to describing the various embodiments, the following definitions are provided and should be used unless otherwise indicated.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim but may contain additional composition components or method steps, etc., that do not materially affect the basic and novel characteristic(s) of the compositions or methods.

“Subject” refers to any animal, preferably a human patient, livestock, rodent, monkey, or domestic pet.

In certain embodiments, methods disclosed herein may make measurements that are compared to a normal or reference value. As used herein, a “reference value” can be an absolute value; a relative value; an average value; a median value, a mean value, or a value as compared to a particular control or baseline value. A reference value can be based on an individual sample or many samples, such as from patients or normal individuals.

A “normalized measured” value refers to a measurement taken and adjusted to take background into consideration. Background subtraction to obtain total fluorescence is considered a normalized measurement. The background subtraction allows for the correction of background fluorescence that is inherent in the optical system and assay buffers.

A “test compound” can be any variety of organic compounds such as small molecules, proteins, antibodies, nucleobases, nucleobase polymers, and known therapeutic agents or therapeutic candidates.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide/disaccharide/polysaccharide, medium chain fatty acids, and/or optionally nucleotides. Typical components in a growth medium include amino acids (histidine, isoleucine, leucine, lysine, methionine, phenylalanine, threonine, tryptophan, valine, and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth medium may contain a pH indicator, e.g., phenol red. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate.

Various growth mediums are known in the art. For example, Minimal Essential Medium (MEM) is a term of art referring to a growth medium that contains calcium chloride, potassium chloride, magnesium sulfate, sodium chloride, sodium phosphate and sodium bicarbonate, essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin M), choline, and inositol (originally known as vitamin B8). Dulbecco's modified Eagle's medium (DMEM) is a growth medium which contains additional components such as glycine, serine, and ferric nitrate with increased amounts of vitamins, amino acids, and glucose. Animal serum such as fetal bovine serum (FBS) is sometimes added to a growth media as a supplement.

“Positron emission tomography” (PET) refers to an imaging technique that produces an image, e.g., three-dimensional image, by detecting pairs of gamma rays emitted indirectly by a positron-emitting radionuclide tracer. Images of tracer concentration within the area are then constructed by computer analysis. A radioactive tracer is administered to a subject e.g., into blood circulation. Typically, there is a waiting period while tracer becomes concentrated in areas of interest: then the subject is placed in the imaging scanner. As the radionuclide undergoes positron emission decay, it emits a positron, an antiparticle of the electron with opposite charge, until it decelerates to a point where it can interact with an electron, producing a pair of (gamma) photons moving in approximately opposite directions. These are detected in a scanning device. The technique typically utilizes simultaneous or coincident detection of the pair of photons moving in approximately opposite direction. Photons that do not arrive in pairs (i.e., within a timing-window) are typically ignored. One typically localizes the source of the photons along a straight line of coincidence (also called the line of response, or LOR). This data is used to generate an image.

The term “radionuclide” or “radioactive isotope” refers to molecules of enriched isotopes that exhibit radioactive decay (e.g., emitting positrons). Such isotopes are also referred to in the art as radioisotopes. A radionuclide tracer does not include radioactive primordial nuclides but does include naturally occurring isotopes that exhibit radioactive decay with an isotope distribution that is enriched, i.e., greater than natural abundance. In certain embodiments. it is contemplated that the radionuclides are limited to those with a half live of less than 1 hour and those with a half-life of more than 1 hour but less than 24 hours. Radioactive isotopes are named herein using various commonly used combinations of the name or symbol of the element and its mass number (e.g., 18F, F-18, or fluorine-18).

Such isotopically labeled compounds are useful in metabolic studies, reaction kinetic studies, detection, or imaging techniques [such as positron emission tomography (PET) or single-photon emission computed tomography (SPECT)] including drug or substrate tissue distribution assays. In particular, an ¹⁸F or ¹¹C labeled compound may be particularly preferred for PET or SPECT studies.

One can produce [¹⁸F] fluoride by irradiation of water (containing H₂¹⁸O) with protons resulting in the ¹⁸O(p,n)¹⁸F reaction. The [¹⁸F] isotope is then separated from water and processed for production of a radiopharmaceutical agent. Typically, fluoride recovery is based on ion exchange resins. Typically, the recovery is carried out in two steps (extraction and elution): first the anions (not only fluoride) are separated from the enriched [¹⁸O] water and trapped on a resin and then, said anions, including [¹⁸F] fluoride, are eluted into a mixture containing water, organic solvents, a base, also called activating agent or phase transfer agent or phase transfer catalyst, such as the complex potassium carbonate-Kryptofix 222™ (K₂CO₃—K₂₂₂) or a tetrabutylammonium salt. Kryptofix 222™ is a cyclic crown ether, which binds the potassium ion, preventing the formation of ¹⁸F—KF. Thus, potassium acts as the counter ion of ¹⁸F⁻ to enhance its reactivity but does not interfere with the synthesis. Typical labeling methods use low water content solutions. An evaporation step may follow the recovery of the [¹⁸F] fluoride, e.g., azeotropic evaporation of acetonitrile or other low boiling temperature organic solvent.

Alternatively, the extraction process is performed by passing the [¹⁸F] aqueous solution on a solid support as reported in U.S. Pat. No. 8,641,903. This solid support is typically loaded with a trapping agent, e.g., compound comprising a quaternary amine that is adsorbed on the solid support and allows the [¹⁸F] activity to be trapped because of its positive charge. The solid support is then flushed with a gas or a neutral solvent to remove or push out most of the residual water. The [¹⁸F] is eluted in an organic solvent or in a mixture of organic solvents and is usable for labelling of precursor compounds.

Certain of the compounds described herein may contain one or more asymmetric centers and may give rise to enantiomers, diastereomers, and other stereoisomeric forms that can be defined, in terms of absolute stereochemistry at each asymmetric atom, as (R)— or(S)— or in terms of the ability to bend plan polarized light in the positive or negative direction. The present chemical entities, compositions and methods are meant to include all such possible isomers, including racemic mixtures, tautomer forms, hydrated forms, optically substantially pure forms, and intermediate mixtures.

### Methods of Use

In certain embodiments, a compound disclosed herein comprising a radionuclide is administered to a subject, and the radionuclide in the subject is used to create an image. The radionuclide can be administered at any suitable dose. The subject can be imaged using any suitable imaging apparatus, for example an apparatus capable of gathering a magnetic resonance image (MRI), a positron emission tomography (PET) scan, or a computed tomography (CT) scan.

In certain embodiments, methods entail administering to a subject (which can be human or animal, for experimental and/or diagnostic purposes) an image-generating amount of a compound of the disclosure, labeled with the appropriate isotope and then measuring the distribution of the compound by PET. An image-generating amount is that amount which is at least able to provide an image in a PET scanner considering the detection sensitivity and noise level of the scanner, the age of the isotope, the body size of the subject and route of administration.

Methods disclosed herein may be combined with other methods such as single photon emission computed tomography (SPECT) scans, computerized tomography (CT) scans, and MRI. A CT scan combines a series of X-ray images taken from different angles uses computer processing to create cross-sectional images, or slices of the brain, bones, blood vessels and soft tissues inside a body. These scans or associated data can be used to create computerized images that take place in tissue. A scanner records data, and a computer constructs two- or three-dimensional images.

It should be noted that the amount effective to result in uptake of the tracer compound into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors.

Preferred imaging methods provided by the present disclosure include the use of the radionuclide containing compounds of the present disclosure and/or salts thereof that can generate at least a 2:1 target to background ratio of radiation intensity, or more preferably about a 5:1, about a 10:1 or about a 15:1 ratio of radiation intensity between target and background. In certain preferred methods, the radiation intensity of the target tissue is more intense than that of the background. In other embodiments, the present disclosure provides methods where the radiation intensity of the target tissue is less intense than that of the background. Generally, any difference in radiation intensity between the target tissue and the background that is sufficient to allow for identification and visualization of the target tissue is sufficient for use in the methods of the present disclosure.

In preferred methods of the present disclosure, the compounds of the present disclosure are excreted from tissues of the body quickly to prevent prolonged exposure to the radiation of the radiolabeled compound administered to the patient. In a particular embodiment, the radionuclide tracer provided herein can be used on an outpatient basis. Typically, compounds of the present disclosure are eliminated from the body in less than about 24 hours.

Images can be generated by virtue of differences in the spatial distribution of the imaging agents that accumulate at a site. The spatial distribution may be measured using any imaging apparatus suitable for the imaging agent, for example, a gamma camera, a PET apparatus, a SPECT apparatus, MRS, MRI, or optical imaging apparatus, and the like. The extent of accumulation of the imaging agent may be quantified using known methods for quantifying radioactive emissions. A particularly useful imaging approach employs more than one imaging agent to perform simultaneous studies. Alternatively, the imaging method may be carried out a plurality of times with increasing administered doses.

The amount of imaging agent used for diagnostic purposes and the duration of the imaging study will depend upon the radionuclide used to label the agent, the body mass of the patient, the nature and severity of the condition being treated, the nature of therapeutic treatments which the patient has undergone, and on the idiosyncratic responses of the patient. Ultimately, the attending physician will decide the amount of imaging agent to administer to each individual patient and the duration of the imaging study.

In a typical method, a radioactive compound is injected into the subject, e.g., through a vein, and a scanner is used to make detailed images of areas inside the body over time where the radioactive material is taken up by the cells, tissue, fluids, or organs. For example, when imaging, the scans for tracking 18F-CHL-2205 are contemplated to show the uptake of this tracer in the spinal cord, brain, brainstem, cerebellum, cortex, hippocampus, midbrain, striatum, thalamus, or combinations thereof.

In certain embodiments, this disclosure relates to imaging methods comprising a) administering a N-cyclopropyl-1-(4-(4-(fluoro-18F)phenyl)pyrimidin-5-yl)-N-methyl piperidine-4-carboxamide tracer to a subject; and b) scanning the subject for the emission/positron-emissions. The methods typically further comprise the steps of detecting the emissions and creating an image of an area of the subject indicating or highlighting the location of the compound containing the radionuclide in the subject. In certain embodiments, the area of the subject is the central nervous system, brain, spinal cord, lymph nodes, groin, axilla, neck, lungs, liver, kidney, pancreas, stomach, balder, intestines, circulatory system, breast, prostate, or gallbladder.

The tracer may be administered by any suitable technique known in the art, such as direct injection. Injection may be an intravenous (IV) injection. Administration may be general or local to the site of interest. The tracer may be used in conjunction with another probe, for example a fluorescent probe. The two (or more) probes may be administered together, separately, or sequentially. The tracer of the present disclosure may be used to diagnose, assess, or monitor the progression or treatment of a disease or condition reported herein.

The tracer of the present disclosure may be used to investigate the effects of a test compound. For example, N-cyclopropyl-1-(4-(4-(fluoro-18F)phenyl)pyrimidin-5-yl)-N-methyl piperidine-4-carboxamide may be administered together with a test compound, to evaluate the effect of the test compound be assayed in real time in vivo using a method in accordance with the present disclosure.

In certain embodiments, this disclosure relates to methods comprising: a) administering a composition comprising the N-cyclopropyl-1-(4-(4-(fluoro-18F)phenyl)pyrimidin-5-yl)-N-methyl piperidine-4-carboxamide tracer isotopically enriched with fluorine 18 to a subject; and scanning the subject for emissions from an area of the subject.

In certain embodiments, the N-cyclopropyl-1-(4-(4-(fluoro-18F)phenyl)pyrimidin-5-yl)-. N-methylpiperidine-4-carboxamide tracer passes the blood brain barrier and emissions are from inside the brain or skull area.

In certain embodiments, methods further comprise the step of detecting and/or measuring the emissions and creating an image indicating or highlighting the location of the compound isotopically enriched with fluorine 18 in the subject.

In certain embodiments, methods comprise the step of detecting, measuring, and/or quantifying the emission providing an emission quantity and optionally correlating the emission measurement/detection/quantity to a concentration of cholesterol 24-hydroxylase (cytochrome P450 46A1) and/or 24S-hydroxycholesterol in the brain or other area, e.g., of the central nervous system.

In certain embodiments, methods further comprise the step of correlating a high or abnormal measurement, quantity, or concentration of cholesterol 24-hydroxylase (cytochrome P450 46A1) and/or 24-hydroxycholesterol to the existence of or diagnosis of a subject at risk of a central nervous system disease or condition. In certain embodiments, the disease is Alzheimer's disease, Huntington's disease, autism, epilepsy, depression, Parkinson's disease, a cognitive or memory disorder, or other neurodegenerative disease.

In certain embodiments, this disclosure relates to method of making a PET imaging agent, i.e., the N-cyclopropyl-1-(4-(4-(fluoro-18F)phenyl)pyrimidin-5-yl)-N-methylpiperidine-4-carboxamide tracer, comprising contacting a precursor compound N-cyclopropyl-N-methyl-1-(4-(4-(4,4,5,5-tetramethyl-1,3,2-dioxaborolan-2-yl)phenyl)pyrimidin-5-yl)piperidine-4-carboxamide with an isotopically enriched fluorine 18 negative ion producing the PET imaging tracer N-cyclopropyl-1-(4-(4-(fluoro-18F)phenyl)pyrimidin-5-yl)-N-methyl piperidine-4-carboxamide.

In certain embodiments, the enriched fluorine 18 negative ion is a fluorine 18 potassium salt bound to a cryptand.

In certain embodiments, methods disclosed herein are used to detect, measure, quantify, assess, diagnose, or evaluate in a subject hydroxycholesterol, or cholesterol 24-hydroxylase concentrations or cholesterol metabolism in a subject suspected of having, being at risk of, or diagnosed with a CNS disorder, neurodegenerative disorder, cognitive disorder, age-and/or sex-related brain disorder.

In certain embodiments, this disclosure relates to methods of administering the N-cyclopropyl-1-(4-(4-(fluoro-18F)phenyl)pyrimidin-5-yl)-N-methylpiperidine-4-carboxamide tracer in combination with CYP46A1-targeted drug or a test compound to a subject and imaging, detecting, measuring, or quantifying the PET signal in a tissue, brain, or portion/segment thereof, of the subject in order to evaluate the ability of a test compound or CYP46A1-targeted drug to bind CYP46A enriched tissue of the subject.

In certain embodiments, methods further comprise determining whether a subject is or would likely be responsive to a CYP46A1-targeted drug or test compound therapy. In certain embodiments, the CYP46A1-targeted drug is voriconazole, thioperamide, or soticlestat. In certain embodiments, the subject has, is suspected of having, at risk of, or diagnosed with a CNS disorder, neurodegenerative disorder, cognitive disorder, impaired memory, or other brain disorder.

In certain embodiments, if the imaging, detecting, measuring, or quantifying of the PET signal indicates the N-cyclopropyl-1-(4-(4-(fluoro-18F)phenyl)pyrimidin-5-yl)-N-methylpiperidine-4-carboxamide tracer binds to the CYP46A enriched tissue of the subject that is lower than a normal or reference value absent the test compound, then said imaging, detecting, measuring, or quantifying provides an indication that the test compound or CYP46A1-targeted drug is binding or likely binding with sufficient affinity to CYP46A1 in the subject to be effective as a potential therapeutic agent.

In certain embodiments, methods further comprise the step of administering an effective amount of the test compound or CYP46A1-targeted drug to a subject in need thereof.

In certain embodiments, if the imaging, detecting, measuring, or quantifying of the PET signal indicates that the N-cyclopropyl-1-(4-(4-(fluoro-18F)phenyl)pyrimidin-5-yl)-N-methylpiperidine-4-carboxamide tracer binds to the CYP46A enriched tissue of the subject is normal when compared to a reference value, then that is an indication that the test compound or CYP46A1-targeted drug is not binding or not binding with sufficient affinity to CYP46A1 in the subject to be effective as a potential therapeutic agent.

In certain embodiments, methods further comprise the step of recording the imaging, detecting, measuring, or quantifying data or diagnostic indications therefrom on a computer readable medium. In certain embodiments, methods further comprise the step of reporting the imaging. detecting, measuring, or quantifying data or diagnostic indications therefrom to a medical professional.

In certain embodiments, this disclosure relates to methods of diagnosing and treating a subject with a neurodegenerative disease or cognitive disorder, condition comprising, administering an effective amount of N-cyclopropyl-1-(4-(4-(fluoro-18F)phenyl)pyrimidin-5-yl)-N-methyl piperidine-4-carboxamide to a subject in need thereof; measuring ¹⁸F brain emissions from the subject; comparing the ¹⁸F brain emission to a normal or reference value; and diagnosing the subject with or at risk of a neurodegenerative disease, or cognitive disorder, if the emission is lower or higher than normal; and treating the subject by administering to the subject an effective amount of a therapeutic agent directed to a neurodegenerative disease or cognitive disorder. In certain embodiments, the therapeutic agent is vector encoding a recombinant CYP46A1.

In certain embodiments, this disclosure relates to a cell culture or growth medium comprising N-cyclopropyl-1-(4-(4-(fluoro-18F)phenyl)pyrimidin-5-yl)-N-methyl piperidine-4-carboxamide or salt thereof.

In certain embodiments, this disclosure relates to evaluating or monitoring the effectiveness of a drug therapy or recombinant therapy comprising administering a drug for treating a neurodegenerative diseases or condition, or a vector encoding a recombinant CYP46A1, e.g., adeno-associated vector therapy, for expression in brain or other CNS tissues, in combination with, or sometime after, the N-cyclopropyl-1-(4-(4-(fluoro-18F)phenyl)pyrimidin-5-yl)-N-methylpiperidine-4-carboxamide tracer, and thereafter imaging, detecting, measuring, or quantifying the tracer at a location in the subject.

In certain embodiments, the subject is a human subject 2, 12, or 16 years old or older or less than 2, 12, or 16 years old. In certain embodiments, the subject is a human subject 55 or 65 years old or older. In certain embodiments, the subject is a human subject greater than 55, 60, 65, or 70 years of age. In certain embodiments, the subject is an infant, e.g., from one month to two years of age. In certain embodiments, the subject is a human subject such as a child, e.g., from two to twelve years of age. In certain embodiments, the subject is a human subject such as an adolescent, e.g., from twelve to sixteen years of age. In certain embodiments, the subject is a human subject sixteen years of age or older.

### Kits

In certain embodiments, this disclosure relates to kits comprising a N-cyclopropyl-1-(4-(4-(fluoro-18F)phenyl) pyrimidin-5-yl)-N-methylpiperidine-4-carboxamide tracer and/or precursor compounds, e.g. N-cyclopropyl-N-methyl-1-(4-(4-(4,4,5,5-tetramethyl-1,3,2-dioxaborolan-2-yl)phenyl)pyrimidin-5-yl)piperidine-4-carboxamide and instructions for use. In certain embodiments, the instructions provide for the activity at the end of synthesis. In certain embodiments, the instructions provide for the half-life of the radionuclide. In certain embodiments, the instructions provide that injection should be used within limited time from the time of the end of synthesis. In certain embodiments, the container is a sealed container such as a septum capped vial.

In certain embodiments, this disclosure relates to kits comprising the precuror N-cyclopropyl-N-methyl-1-(4-(4-(4,4,5,5-tetramethyl-1,3,2-dioxaborolan-2-yl)phenyl)pyrimidin-5-yl)piperidine-4-carboxamide and starting materials to make the radionuclide tracer N-cyclopropyl-1-(4-(4-(fluoro-18F)phenyl)pyrimidin-5-yl)-N-methyl piperidine-4-carboxamide, and/or a substance for preparing a radionuclide in a cyclotron. In certain embodiments, the kit comprises a container having water, H₂¹⁸O, and/or ethanol in water solution. In certain embodiments, the container is sealed from the atmosphere. In certain embodiments, kits comprise a solid support or filter. In certain embodiments, the filter may be used to purify a radionuclide tracer disclosed herein.

In certain embodiments, the N-cyclopropyl-1-(4-(4-(fluoro-18F)phenyl)pyrimidin-5-yl)-N-methylpiperidine-4-carboxamide tracer may be prepared at the location of the subject near the time the subject is exposed to an imaging device. Thus, in certain embodiments, the disclosure contemplates kits comprising the precursor compound N-cyclopropyl-N-methyl-1-(4-(4-(4,4,5,5-tetramethyl-1,3,2-dioxaborolan-2-yl)phenyl)pyrimidin-5-yl)piperidine-4-carboxamide and a solid support.

In some embodiments, the solid support is selected from the group of solid phase extraction resins or liquid chromatography resins, e.g., silica (oxide) based or non-silica (metal oxide or polymers) based particles optionally functionalized (e.g., by organosilanization) with alkyl chains for example C4, C8, C18, C30 or other functional groups, e.g., polar groups (amide, carbamate, and urea) embedded within alkyl chains or branched alkyl groups or polymeric packings.

In some embodiments, the solid support is selected from the group consisting of solid phase extraction resins and liquid chromatography resins resulting from the copolymerization of divinylbenzene and/or styrene, or by the copolymerization with vinylpyrrolidone, vinylacetate, (methacryloyloxymethyl)naphtalene, 4,4′-bis(maleimido)diphenylmethane, p,p′-dihydroxy diphenylmethane diglycidylmethacrylic ester, p,p′-dihydroxydiphenylpropane diglycidylmethacrylic ester, 2-hydroxyethylmethacrylate (HEMA), 2,2-dimethylaminoethylmethacrylate (DMAEMA), ethylenedimethacrylate glycidylmethacrylate, N-vinylcarbazole, acrylonitrile, vinylpyridine, N-methyl-N-vinylacetamide, aminostyrene, methylacrylate, ethylacrylate, methylmethacrylate, N-vinylcaprolactam, N-methyl-N-vinylacetamide.

In some embodiments, the solid support comprises or is functionalized with or preconditioned with:
quaternary ammonium salts, e.g., tetraethylammonium carbonate, tetrabutylammonium carbonate;
potassium carbonate cryptands such as [2.2.2] cryptand N(CH₂CH₂OCH₂CH₂OCH₂CH₂)₃N, 1,4,10-trioxa-7,13-diaza-cyclopentadecane, 4,7,13,16,21,24-hexaoxa-1, 10-diazabicyclo[8.8.8]hexacosane, 4,7,13,16,21-pentaoxa-1,10-diazabicyclo[8.8.5]tricosane, 4,7,13,18-tetraoxa-1,10-diazabicyclo[8.5.5] eicosane, 5,6-benzo-4,7,13,16,21,24-hexaoxa-1,10-diazabicyclo[8.8.8]hexacos-5-ene;
crown ethers such as 4′-aminobenzo-15-crown-5, 4′-aminobenzo-15-crown-5, 4′-aminobenzo-15-crown-5 hydrochloride, 4′-aminobenzo-18-crown-6, a′-Aminodibenzo-18-crown-6, 2-aminomethyl-15-crown-5, 2-aminomethyl-15-crown-5, 2-aminomethyl-18-crown-6, 4′-amino-5′-nitrobenzo-15-crown-5, 4′-amino-5′-nitrobenzo-15-crown-5, 1-aza-12-crown-4, 1-aza-15-crown-5, 1-aza-15-crown-5, 1-aza-18-crown-6, 1-aza-18-crown-6, benzo-12-crown-4, 5,6-benzo-4,7,13,16,21,24-hexaoxa-1, 10-diazabicyclo[8.8.8]hexacos-5-ene, 1-benzyl-1-aza-12-crown-4, bis[(benzo-15-crown-5)-15-ylmethyl]pimelate, 4′-bromobenzo-15-crown-5, 4-tert-butylbenzo-15-crown-5, 4-tert-butylcyclohexano-15-crown-5, 4′-carboxybenzo-15-crown-5;
calixarenes such as 4-tert-butylcalix[4]arene, 4-tert-butylcalix[4]arene, 4-tert-butylcalix[4]arene, 4-tert-butylcalix[5]arene, 4-tert-butylcalix[6]arene, 4-tert-butylcalix[6]arene, 4-tert-butylcalix[6]arene, 4-tert-butylcalix[8]arene, 4-tert-butylcalix[8]arene, 4-tert-butylcalix[4]arene-tetraacetic acid tetraethyl ester, 4-tert-butylcalix[4]arenetetraacetic acid tetraethyl ester, 4-tert-butylcalix[4]arene-tetraacetic acid triethyl ester, calix[4]arene, calix[6]arene, calix[8]arene, 4-(chloromethyl)calix[4]arene, 4-isopropylcalix[4]arene, C-methylcalix[4]resorcinarene, C-methylcalix[4]resorcinarene, meso-octamethylcalix (4) pyrrole, 4-sulfocalix[4]arene, 4-sulfocalix[4]arene sodium salt, C-undecylcalix[4]resorcinarene monohydrate, C-undecylcalix[4]resorcinarene monohydrate;
cyclodextrines such as α-cyclodextrin, β-cyclodextrin, γ-cyclodextrin, (2,6-di-O-) ethyl-β-cyclodextrin, 6-O-α-D-glucosyl-β-cyclodextrin, heptakis(6-O-t-butyldimethylsilyl-2,3-di-O-acetyl)-β-cyclodextrin, heptakis(2,6-di-O-methyl)-β-cyclodextrin, heptakis(2,3,6-tri-O-acetyl)-β-cyclodextrin, heptakis(2,3,6-tri-O-benzoyl)-β-cyclodextrin, hexakis (6-O-tertbutyl-dimethylsilyl)-α-cyclodextrin, hexakis (2,3,6-tri-O-acetyl)-α-cyclodextrin, hexakis (2,3,6-tri-O-methyl)-α-cyclodextrin, (2-hydroxyethyl)-β-cyclodextrin, 6-O-α-maltosyl-β-cyclodextrin hydrate, methyl-β-cyclodextrin, 6-monodeoxy-6-monoamino-β-cyclodextrin, octakis (6-O-t-butyldimethylsilyl)-γ-cyclodextrin, sulfopropyl-β-cyclodextrin, triacetyl-α-cyclodextrin, triacetyl-β-cyclodextrin;
EDTA and derivatives such as ethylenediamine-N,N′-diacetic acid, 2-bis(2-aminophenoxy)ethane-N,N,N′,N′-tetraacetic acid, trans-1,2-diaminocyclohexane-N,N,N′,N′-tetraacetic acid monohydrate, trans-1,2-diaminocyclohexane-N,N,N′,N′-tetraacetic acid monohydrate, 1,3-diamino-2-hydroxypropane-N,N,N′,N′-tetraacetic acid, 1,2-diaminopropane-N,N,N′,N′-tetraacetic acid, 1,3-diaminopropane-N,N,N′,N′-tetraacetic acid, 1,3-diamino-2-propanol-N,N,N′,N′-tetraacetic acid, diethylenetriamine-pentaacetic acid calcium trisodium salt hydrate, N-(2-hydroxyethyl) ethylenediaminetriacetic acid trisodium salt hydrate, N-(2-hydroxyethyl)ethylenediamine-N,N′,N′-triacetic acid; and/or
polyethylene glycols (PEG), polyethylene oxides (PEO).

### Pharmaceutical Compositions

In certain embodiments, this disclosure relates to pharmaceutical compositions comprising N-cyclopropyl-1-(4-(4-(fluoro-18F)phenyl)pyrimidin-5-yl)-N-methylpiperidine-4-carboxamide or salt thereof or a precursor compound disclosed herein such as N-cyclopropyl-N-methyl-1-(4-)4-(4,4,5,5-tetramethyl-1,3,2-dioxaborolan-2-yl)phenyl)pyrimidin-5-yl)piperidine-4-carboxamide or salt thereof or and a pharmaceutically acceptable excipient.

In certain embodiments, the disclosure relates to a pharmaceutical composition comprising a compound as described herein including salts thereof and a pharmaceutically acceptable excipient, diluent, or carrier. In certain embodiments, the pharmaceutical composition is in the form of a powder, liquid, or aqueous buffered solution. In certain embodiments, the buffered solution is a citrate buffered solution, isotonic solution, sterile solution, pyrogen free solution, endotoxins and exotoxins free solution, lipopolysaccharide free solution, and/or bacterial free solution.

Pharmaceutical compositions disclosed herein may be in the form of pharmaceutically acceptable salts. Some preferred, but non-limiting examples of pharmaceutically acceptable acids for salt formation are hydrochloric acid, hydrobromic acid, sulfuric acid, nitric acid, acetic acid, ascorbic acid, and citric acid, as well as other pharmaceutically acceptable acids known per se.

### Assessment of Cholesterol Homeostasis in the Living Human Brain

A CYP46A1-targeted positron emission tomography (PET) tracer was developed and tested in animal models and in humans. The probe provides accurate noninvasive quantification of CYP46A1 abundancy and cholesterol metabolism across different regions of the rodent, nonhuman primate (NHP), and human brain. The tracer is sensitive to differences in brain cholesterol metabolism between mouse model of Alzheimer's disease (3xTg-AD mice) and wild-type animals. Furthermore, there is preliminary evidence for considerable differences in brain cholesterol metabolism between healthy women and men undergoing CYP46A1-targeted PET imaging.

Alterations in brain cholesterol homeostasis are implicated in neurological disorders. Excess neuronal cholesterol is primarily eliminated by metabolic clearance via cytochrome P450 46A1 (CYP46A1). Methods for visualizing cholesterol metabolism in the living human brain are contemplated. There is a need for a noninvasive technology that quantitatively measures the extent of brain cholesterol metabolism via CYP46A1 to evaluate the effectiveness of treatment options using targeted therapies.

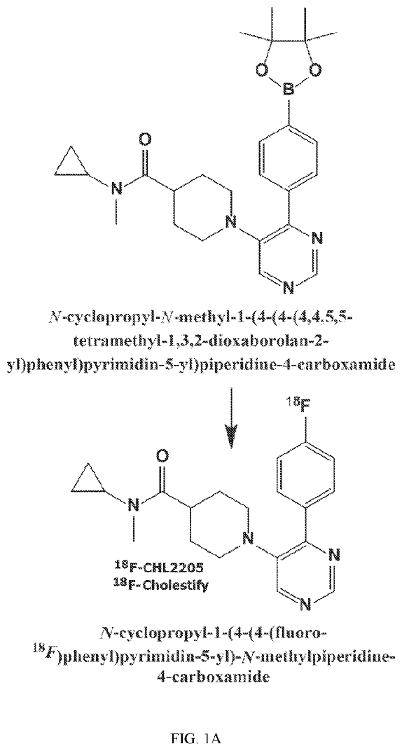

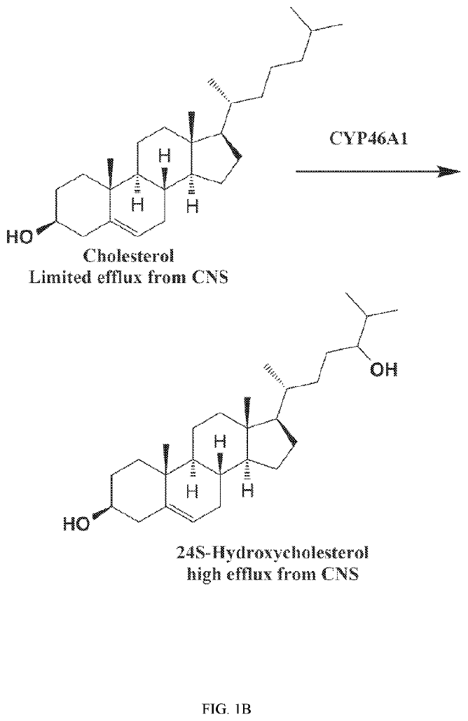

A CYP46A1-targeted positron emission tomography (PET) tracer, 18F-CHL-2205 (18F-Cholestify, FIG. 1A) was developed and tested. PET imaging readouts correlate with CYP46A1 protein expression and with the extent to which cholesterol is metabolized in the brain. In vivo efficacy is provided in the well-established 3xTg-AD murine model of Alzheimer's disease (AD), where experiments indicate that the probe is sensitive to differences in brain cholesterol metabolism between 3xTg-AD mice and control animals. Experiments indicate that there is a considerably higher baseline brain cholesterol clearance via CYP46A1 in women, as compared to age-matched men. These findings illustrate the ability to assess brain cholesterol metabolism using PET and establish PET with 18F-CHL-2205 as a sensitive tool for noninvasive assessment of brain cholesterol homeostasis in the clinic.

Experiments reported herein indicate that one can use 18F-CHL-2205 in the noninvasive in vivo quantification of brain abundancy and activity of CYP46A1, the primary enzyme responsible for clearance of cholesterol from the mammalian CNS. As a PET tracer for CYP46A1, experiments indicate that molecular imaging readouts correlate with CYP46A1 protein expression and the extent to which brain cholesterol is metabolized to 24-hydroxycholesterol. Data indicate differences in CYP46A1-related tracer uptake between a mouse model of AD (3xTg-AD) and wild-type animals. Certain experiments indicate differences in brain cholesterol metabolism via CYP46A1 between women and men.

The proper balance between cholesterol biosynthesis and clearance is a feature of the healthy mammalian CNS. Impaired neuronal cholesterol homeostasis is believed to be linked to several common neurological disorders including AD. Small-animal PET imaging indicates an enhanced CYP46A1-related signal in the brains of 3xTg-AD mice, as compared to control animals. It is contemplated that CYP46A1 function is enhanced in early AD, as an attempt to eliminate excess neuronal cholesterol and attenuate further accumulation of amyloidogenic cholesterol esters. Patients with advanced AD tend to present with blunted concentrations of 24-hydroxycholesterol, potentially owing to the loss of neurons in CYP46A1-expressing brain areas. Although degeneration of neurons is less pronounced in mouse models of AD compared to patients, these observations raise concerns about the interpretation of CYP46A1 protein measurements in AD patients, particularly those with advanced disease stages. To assess probe specificity, validated CYP46A1 inhibitors such as voriconazole and soticlestat were also tested.

Studies reported herein demonstrate the specificity and selectivity of PET tracer 18F-CHL-2205 for CYP46A1. 18F-CHL-2205 can be used as a tool for the assessment of CYP46A1 abundancy in humans and constitutes a major step forward because it enables mechanistic studies on brain cholesterol metabolism in AD and other conditions. Experiments indicate a generally high tracer uptake in the brain, with persistently high uptake only in CYP46A1-rich arcas. Although most high-expression regions were conserved across species, the hippocampus constituted a notable exception. This area was among the high-expression regions in rodents, whereas lower CYP46A1 expression was noted in NHPs and humans. The effectiveness of CYP46A1-targeted PET as a surrogate marker of cholesterol metabolism provides for use as an imaging biomarker for impaired cholesterol homeostasis and for assessing responses to therapy.

### PET Tracer Colocalizes with CYP46A1 and 24-Hydroxycholersterol in the Rodent Brain

Experiments were performed to determine whether a targeted PET imaging probe would allow the quantification of cholesterol metabolism in the living brain, ultimately paving the way for mechanistic studies on brain cholesterol metabolism in humans. A highly potent ligand for CYP46A1 was labeled with fluorine-18 via the boronic pinacol ester precursor compound, N-cyclopropyl-N-methyl-1-(4-(4-(4,4,5,5-tetramethyl-1,3,2-dioxaborolan-2-yl)phenyl)pyrimidin-5-yl)piperidine-4-carboxamide (FIG. 1A). The resulting PET tracer, 18F-CHL-2205 (named 18F-Cholestify), exhibited a selectivity for CYP46A1-rich areas of the rodent brain by in vitro autoradiography. Highest tracer binding was observed in the CYP46A1-rich cortical, hippocampal, striatal, and thalamic structures, whereas lower abundancy was detected in the cerebellum. The tracer distribution matched closely with the Allen Brain Atlas distribution of CYP46A1 expression data in the mouse brain. Quantification of CYP46A1 by Western blot analysis in the same brain regions confirmed that tracer binding patterns were in concert with CYP46A1 expression across all tested brain regions. Saturation binding studies were performed to determine the dissociation constant (KD) of the probe as well as to assess the amount of available CYP46A1 protein (Bmax) in selected brain regions. Bmax values ranged from 117.3 fmol/g in the striatum to 21.9 fmol/g in the cerebellum, and a subnanomolar KD value of 0.36 nM was obtained using striatal brain homogenates. Furthermore, pharmacological screening on major CNS targets, including common G protein-coupled receptors, ion channels, enzymes, and transporters, at a testing concentration of 10 μM revealed no off-target activity of the target compound, 18F-CHL-2205.

Cholesterol interacts with the orthosteric CYP46A1 binding site, triggering its conversion to 24S-hydroxycholesterol in the mammalian brain (FIG. 1B). To define the molecular interactions between 18F-CHL-2205 and CYP46A1, docking studies were conducted using reported crystal structures of CYP46A1. Of the available crystal structures, PDB:3MDT, PDB:7LRL and PDB:3MDM were used. When F-CHL-2205 was docked to CYP46A1, the binding pose resembled that of voriconazole, thioperamide, and soticlestat, indicating that F-CHL-2205 constitutes an orthosteric ligand, which is in accordance with experimental findings showing competitive displacement of 18F-CHL-2205 by voriconazole and soticlestat.

18F-CHL-2205 was stable against radio defluorination when incubated with mouse, rat, VHP, and human liver microsomes. PET imaging studies were conducted by intravenous injection of 18F-CHL-2205 to rats. Systemic administration resulted in a rapid tracer uptake into the brain—with similarity between in vitro and in vivo tracer binding patterns. Greater than 99% of the brain signal was attributed to the intact parent tracer. The brain signal was substantially devoid of interference by radiometabolites. By ex vivo studies, greater than 90% of the signal in the brain was specific at 15, 30, and 45 min after injection. Dosimetry experiments revealed an effective dose of 0.012 mSv/MBq, and PET studies in mice corroborated the probe selectivity for the CYP46A1-rich striatum. These results indicated that 18F-CHL-2205 can be used in PET to visualize CYP46A1 in vivo. Overall, in vitro autoradiography and ex vivo biodistribution studies suggested excellent specificity of 18F-CHL-2205 toward CYP46A1.

Cell uptake studies were conducted with transfected human embryonic kidney (HEK) cells overexpressing human CYP46A1 (hCYP46A1) and respective controls. Transfected cells displayed a fivefold increase in cell uptake, as compared to the controls, which was observed at early incubation time points and did not change over time. Furthermore, the tracer signal of the transfected cell population was attenuated in a dose-dependent manner by co-incubation with escalating doses of soticlestat.

A consideration in CNS-targeted PET constitutes the ability of the tracer to selectivity bind to a target protein while lacking interactions with other abundant brain proteins. To assess whether 18F-CHL-2205 exhibited such selectivity, the brain binding patterns between Cyp46A1 knockout (Cyp46A1−/−) and respective control (Cyp46A1+/+) mice were compared. High tracer binding was observed in the control brain, whereas the signal diminished in the brains of Cyp46A1−/− animals. A blockade experiment with an excess dose of the CYP46A1 inhibitor, soticlestat, showed that the tracer binding in control brains was reduced to the signal observed in Cyp46A1−/− animals, corroborating that the tracer is selectivity for CYP46A1.

To assess whether PET imaging findings reflected not only the abundancy of CYP46A1 but also the extent to which neuronal cholesterol was metabolized to 24-hydroxycholesterol in distinct brain regions, the amount of 24-hydroxycholesterol was measured by advanced mass spectrometry. There was a strong correlation between the PET signal in vivo and amount of 24-hydroxycholesterol ex vivo across all tested brain regions, suggesting that the PET scan can be used to quantify the metabolic activity of CYP46Al in the living brain. When the same experiment was conducted with a related brain cholesterol metabolite, 24,25-epoxycholesterol, that is formed via CYP46A1-dependent and CYP46A1-independent enzymatic reactions, the correlation was lost. These results indicate that the PET signal serves as a noninvasive surrogate measure for the extent of cholesterol turnover to 24-hydroxycholesterol across different brain regions.

Tracer binding was assessed and validated in the 3xTg-AD mouse model. This model is widely used and exhibits some similarities with histopathological and behavioral features of clinical AD. Hippocampal uptake of the CYP46A1 tracer was compared between 3xTg-AD mice and respective controls. After intravenous administration of the CYP46A1 tracer, time-activity curves (TACs) were consistently higher in the hippocampus of 3xTg-AD mice than in controls, pointing toward an increased metabolic clearance of brain cholesterol by CYP46A1 in the AD model (FIG. 1C). When CYP46A1 binding sites were presaturated (blocked) with excess nonradioactive CHL-2205, tracer uptake in 3xTg-AD and control mice was reduced to the same baseline signal, supporting the notion that differences in tracer uptake were attributed to distinct bioavailability of CYP46A1.

### Targeted PET Predicts CYP46A1 Abundancy and Allows the Quantification of Drug-CYP46A1 Interactions in the Brain

PET imaging experiments were conducted in non-human primates (NHPs). Consistent with observations from rodent data, the signal intensity of tracer binding correlated with high abundancy regions for CYP46A1, including the putamen, caudate, and limbic cortical regions such as superficial layers of temporal, insular, and cingulate cortex, as evidenced by in vitro autoradiograms of the brain. By co-incubation with excess soticlestat, a relative signal reduction was observed suggesting that the tracer is specific toward CYP46A1 in NHPs.

The tracer 18F-CHL-2205 was injected intravenously to rhesus monkeys, and the TACs in the brain were recorded by PET over a scan duration of 90 min. Regional brain distributions were consistent between in vitro autoradiograms and in vivo PET. The highest tracer uptake was detected in the putamen, caudate, and cortical regions, followed by the thalamus and hippocampus. The lowest tracer uptake was detected in the cerebellum, brainstem, and white matter regions, implying that these regions exhibited pour CYP46A1 abundancy

To test whether targeted visualization and quantitative assessment of drug-CYP46A1 interactions in the brain of NHPs could be obtained with this PET tracer, a dose-response experiment was performed using soticlestat as a model compound. Using a broad array of soticlestat intravenous doses, ranging from 0.04 to 0.9 μmol/kg, a consistent dose-dependent PET signal attenuation was observed, which allowed target occupancy modeling with noteworthy model accuracy across different brain regions. D₅₀ values (dose that exhibits 50% target occupancy) were around 0.1 μmol/kg for the hippocampus, striatum (caudate/putamen), and prefrontal cortex, with a target occupancy of greater than 90% achieved at the dose of 0.9 μmol/kg for all regions. These results demonstrate that 18F-CHL-2205 PET serves as a valuable tool to assess the extent to which CYP46A1 inhibitors engage with their target at a given dose regimen.

A major consideration of neuroimaging is that PET image reflects a balance between multiple dynamic molecular processes that may include tracer delivery to the brain, binding to the desired target protein, potential metabolism or cellular internalization, and washout from the brain. To assess the extent to which the PET signal reflected tracer binding to CYP46A1, kinetic modeling was performed and tissue volumes of distribution (VT) and nondisplaceable binding potentials (BP_{ND}) were calculated from a two-tissue compartment model. VT constitutes the ratio of tracer concentrations in the target region versus in the plasma at equilibrium, whereas BP_{ND} can be considered a quantitative index of in vivo target abundancy. Using kinetic modeling, VT values across different brain regions revealed a comparable pattern to what was observed from PET images. These findings were further corroborated by a strong correlation between the PET signal and kinetic modeling parameters. Similarly, the PET signal correlated with the expression of CYP46A1, as assessed by Western blot analysis of postmortem brain tissue (PET versus Western blot analysis). These results suggest that PET imaging reflects the abundancy of CYP46A1 across different brain regions of NHPs.

### Quantitative Assessment of Cholesterol Metabolism in the Living Human Brain

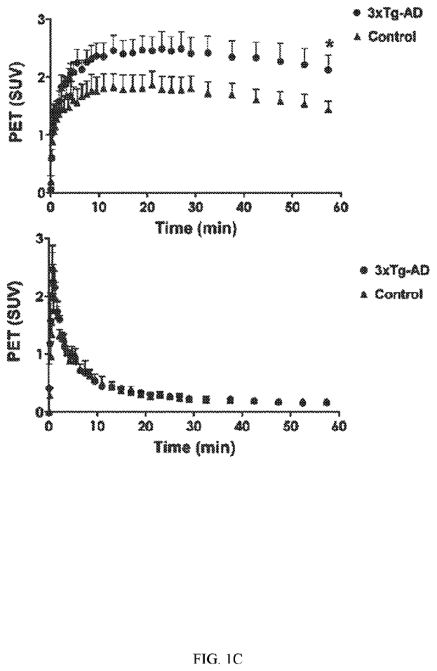

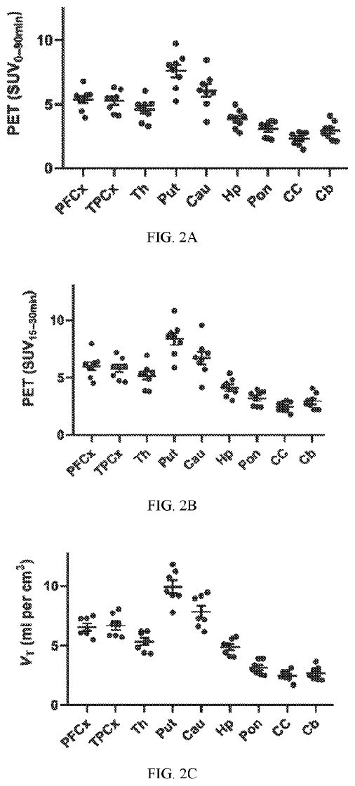

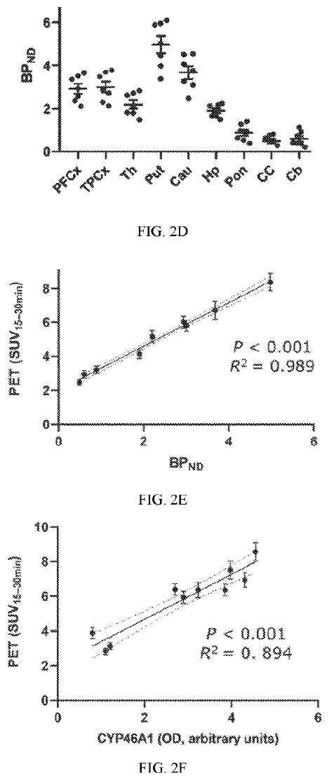

Eight healthy participants underwent a PET scan with 18F-CHL-2205, followed by a magnetic resonance imaging (MRI) scan for anatomical orientation. Because of the physical half-life of fluorine-18 (109.8 min), the tracer was produced on the day of the experiment in a designated hot cell that provided shielding from radiation exposure and was equipped with an automated synthesis module. PET scans were performed for a duration of 90 min, and the data were subsequently reconstructed to allow the assessment of tracer uptake as a surrogate for CYP46A1 abundancy and cholesterol metabolism. Averaged standardized uptake values for a PET scan duration of 90 min (SUV0-90) revealed a heterogeneous tracer accumulation pattern in the human brain, with areas of high radioactivity in the cortical, thalamic, and basal ganglia regions. In contrast, brain areas with limited CYP46A1 abundancy such as the cerebellum, brainstem, and corpus callosum revealed the lowest tracer accumulation (FIG. 2A). Hence, the distribution and in vivo binding of 18F-CHL-2205 in the human brain closely matched the binding patterns in the rodent and NHP brain.

For patients undergoing PET imaging, scan duration of 90 min constitutes a major challenge because minor head movements may prompt detrimental effects on the image quality. To shorten the required scan duration in future studies, experiments were performed to determine whether averaged standardized uptake values (SUVs) from 15 to 30 min after tracer injection (SUV15-30) proved useful for quantitative PET. A comparison between SUV15-30 with regional tissue volumes of distribution and binding potentials revealed a virtually identical distribution pattern across a broad range of tested brain regions (FIG. 2B-2D). Data indicated a strong relationship between SUV15-30 and non-displaceable binding potentials (BPND), which was comparable to that of SUV0-90 versus BP_{ND} (FIG. 2E). These findings indicated that targeted PET accurately predicted the bioavailability of CYP46A1 in humans, with a relatively short scan duration of 15 min. To provide further evidence corroborating that quantitative PET constitutes an accurate surrogate measure of CYP46A1 tissue density in various brain regions, Western blot analysis was performed with postmortem specimens of healthy human brains. Quantification of CYP46A1 by Western blot analysis revealed high target protein expression in the caudate, putamen, and various cortical regions, whereas lowest CYP46A1 abundancy was found in the cerebellum, pons, and white matter. The strong relationship observed between PET and Western blot analysis across various brain areas (FIG. 2F) further indicates that the PET signal constitutes an accurate noninvasive measure of CYP46A1 protein density, providing a valuable tool to assess cholesterol metabolism in the living human brain.

Experiments were performed to determine whether CYP46A1-targeted PET was sensitive to sex differences in brain cholesterol metabolism. Women generally showed a higher brain PET signal, and the regional values of SUV15-30 in the caudate and putamen (CYP46A1-rich) regions were higher in women than in men. Conversely, there was no difference in tracer uptake for low-target density regions such as the cerebellum and brainstem. These findings imply that considerable sex differences in brain cholesterol metabolism via CYP46A1 exist in humans.

### Cell Uptake Studies

Cell uptake assays were conducted using hCYP46A1 enzyme-HEK cell line (HEK-CYP46A1) and HEK control cells were cultured in Dulbecco's modified Eagle's medium (DMEM) supplemented with 10% fetal bovine serum and antibiotics (0.1 mg/ml streptomycin and 100 IU/ml penicillin). Cells were kept under standard conditions at 37° C. and 5% CO₂ and subsequently subpassaged using a solution of 0.25% trypsin and 0.02% EDTA. For cell uptake measurements, HEK-CYP46A1 and HEK control cells were seeded into a 24-well plate at a density of 2×10⁵ cells per well the day before the experiment. The medium was replaced with fresh DMEM including ¹⁸F-CHL-2205 (ca. 74 kBq per well) and incubated at 37° C. for 15, 30, and 45 min. The supernatant was collected, and the cells were washed twice with cold 1×phosphate-buffered salinc (PBS) and then harvested by adding 200 μl of 1N NaOH followed by additional two times rinsing with 1×PBS. The blocking assay was performed with the HEK-CYP46A1 cells. The nonradioactive compound, soticlestat, was added in various concentrations. The incubation time was 1 hour, and the washing steps were repeated once. Supernatant and cell suspensions including 1×PBS from each wash step were collected and then measured using a gamma counter, whereas cell uptake was expressed as the percentage of the added dose after decay correction.

### Sterol Measurements

Sterol measurements were conducted using frozen tissue sections (10 μm thickness) on glass slides that were dried in a vacuum desiccator. A mixture of isotope-labeled standards was sprayed onto the tissue followed by cholesterol oxidase (0.264 U/ml) in 50 mM KH₂PO₄ (pH 7). The samples were incubated at 37° C. for 1 hour in a damp atmosphere, after which the slides were dried in a vacuum desiccator. [²H₅] Girard P reagent (6.3 mg/ml of bromide salt) in 70% methanol and 5% acetic acid was sprayed on the dried tissue, which was then incubated in a methanol/water atmosphere at 37° C. for 1 hour. The slides were removed and dried before analysis by liquid extraction for surface analysis (LESA)-liquid chromatography-mass spectrometry (LC-MS).

### PET Imaging Studies in Naïve Rodents and 3×Tg-AD Mice

CD1, 3×Tg-AD mice and respective controls (sex: female, age: 9 to 13 months), as well as rats were purchased from established commercial vendors and kept under a 12-hour light/12-hour dark cycle, with ad libitum food and water. Animals were allowed to acclimatize for at least 1 week before the start of the experimental procedures. Mice and rats were anesthetized with isoflurane and scanned in a μPET/computed tomography (CT) scanner for 60 min after tail-vein injection of 18F-CHL-2205. Data were reconstructed in user-defined time frames. Time-activity curves (TACs) were calculated with predefined regions of interest. Results are presented as area under the curve (AUC) from the respective TACs of SUVs, indicating the decay-corrected radioactivity per cubic centimeter, divided by the injected dose per gram of body weight.

### PET/CT and MRI Studies in Rhesus Monkeys

Four rhesus monkeys (4.8 to 9.3 kg) were anesthetized by intramuscular injection of ketamine (10 mg/kg) and then put on the MRI scanning bed in the supine position. Whole-brain images were acquired. For PET, the subjects were initially anesthetized with ketamine (10 mg/kg, intramuscularly), put on the scanning bed, and maintained under anesthesia with 2% isoflurane and 98% oxygen. All rhesus monkeys were supine, and a stereotactic frame was used to fix the position of the head. A bolus intravenous injection of 18F-CHL-2205 (104 to 176 MBq, 0.38 to 1.49 μg) was performed into the monkey through an intravenous catheter, followed by a dynamic PET scan of the head for 90 min. For target occupancy studies, the CYP46A1 inhibitor soticlestat was administered intravenously at doses of 0.0009, 0.0016, 0.014, 0.016, 0.05, 0.12, 0.32, and 0.34 mg/kg, followed by the injection of 18F-CHL-2205. PET/CT and MR images were co-registered. TACs were derived from the respective volumes of interest and were presented as SUVs, which were decay-corrected to the time of radioligand injection. Whole-blood samples were collected from an arterial catheter to measure radioactivity in whole blood and plasma. Samples were centrifuged, and 100 μl of whole blood and plasma was measured in a gamma counter at 10, 20, 30, 40, 50, 60, 75, 90, 105, 120, 150, 180, 210, 240, 600, 900, 1200, 1800, 2700, 3920, 4500, and 5400 s after 18F-CHL-2205 injection. Plasma radiometabolite analysis was performed by collecting additional arterial blood samples (1.0 to 2.5 ml) at 2, 10, 30, 60, and 90 min after injection. These samples were then centrifuged at 6500 rpm at 4° C. for 5 min. Portions of the plasma (ca. 0.4 ml) were deproteinated using the same volume of ice-cold acetonitrile, vortexed, and separated by centrifugation for 3 min at 14,500 rpm and 4° C. The supernatant was mixed with 0.02 ml of CH₃CN in which the reference compound CHL-2205 (ca. 0.02 mg) was dissolved.

Then, the mixture was injected into a high-performance liquid chromatography (HPLC) system equipped with a semipreparative IIPLC column and an ultraviolet detector with the wavelength set at 254 nm. The mobile phase consisted of acetonitrile/water (9:1, v/v), and the flow rate was 2.0 ml/min. The HPLC fractions were collected at 30-s intervals for 13 min; each fraction was counted with automatic gamma well counter. The results were corrected for background radiation and physical decay, whereas unmetabolized 18F-CHL-2205 parent fraction was determined as the ratio of the sum of radioactivity in fractions containing the parent (reference) compound to the total amount of radioactivity collected. Tissue volumes of distribution (VT) and non-displaceable binding potential (BP_{ND}) values were determined from a two-tissue compartment model, with metabolite-corrected arterial input function.

### Clinical Study

An open-label clinical study to evaluate the tolerability, safety, and tracer performance characteristics of 18F-CHL-2205 in healthy volunteers (four men and four women, 22 to 31 years of age) was conducted. Prespecified outcomes of this study included tracer kinetics and distribution volumes of 18F-CHL-2205 in the brain, adverse events for up to 10 days after intravenous bolus administration of the tracer. All subjects underwent a dynamic 90-min PET and MRI scan after injection of 18F-CHL-2205 (173.9 to 305.8 MBq. 1.57 to 3.63 μg). Individual MRI images were used for anatomical orientation and to delineate volumes of interest. Vital sign and electrocardiogram records were taken just before and after the scans. Two-tissue compartment model analyses were conducted using image-derived input functions from the carotid arteries, which provided excellent model fits. Volumes of distribution were further determined by Logan graphical analysis and were used to calculate binding potentials. One individual was excluded from kinetic modeling because of limited image quality for the carotid artery PET data are presented as SUVs, averaged from 0 to 90 min (SUVO 90) or from 15 to 30 min (SUV15 30) after injection.

What is claimed is:

1. A compound N-cyclopropyl-1-(4-(4-(fluoro-18F)phenyl)pyrimidin-5-yl)-N-methyl piperidine-4-carboxamide or salt thereof.

2. A pharmaceutical composition comprising N-cyclopropyl-1-(4-(4-(fluoro-18F)phenyl)pyrimidin-5-yl)-N-methylpiperidine-4-carboxamide or salt thereof and pharmaceutically acceptable excipient.

3. A compound N-cyclopropyl-N-methyl-1-(4-(4-(4,4,5,5-tetramethyl-1,3,2-dioxaborolan-2-yl)phenyl)pyrimidin-5-yl)piperidine-4-carboxamide or salt thereof.

4. A kit comprising a compound as in claim 3.

5. The kit of claim 4 further comprising potassium bound to a cryptand.

6. A method of making a compound N-cyclopropyl-1-(4-(4-(fluoro-18F)phenyl)pyrimidin-5-yl)-N-methylpiperidine-4-carboxamide comprising
contacting a compound comprising N-cyclopropyl-N-methyl-1-(4-(4-(4,4,5,5-tetramethyl-1,3,2-dioxaborolan-2-yl)phenyl)pyrimidin-5-yl)piperidine-4-carboxamide with an isotopically enriched fluorine 18 negative ion producing the compound N-cyclopropyl-1-(4-(4-(fluoro-18F)phenyl)pyrimidin-5-yl)-N-methyl piperidine-4-carboxamide.

7. The method of claim 6 wherein the enriched fluorine 18 negative ion is a fluorine 18 potassium salt bound to a cryptand.

8. A method comprising:
a) administering a composition comprising the compound N-cyclopropyl-1-(4-(4-(fluoro-18F)phenyl)pyrimidin-5-yl)-N-methyl piperidine-4-carboxamide or salt thereof isotopically enriched with fluorine 18 to a subject; and
b) scanning the subject for emissions from an area of the subject.

9. The method of claim 8, wherein the emissions are from inside the brain.

10. The method of claim 8, wherein administering is at a dose of 0.04 to 0.9 μmol/kg.

11. The method of claim 8, wherein scanning the subject for emissions is in the brain of the subject for between 30 and 15 min.

12. The method of claim 8, wherein administering is at a dose of 0.9 μmol/kg and scanning the subject for emissions is in the brain for 15 min.

13. The method of claim 8 further comprising the step of detecting the emissions and creating an image indicating or highlighting the location of the compound isotopically enriched with fluorine 18 in the subject.

14. The method of claim 8 further comprising the step of quantifying the emission providing an emission quantity and recording the emission quantity on computer readable media.

15. The method of claim 14 further comprising correlating the emission quantity to concentration of cytochrome P450 46A1 and/or 24-hydroxycholesterol in the brain or cerebrospinal fluid.

16. The method of claim 15 further comprising reporting the emission quantity to a medical professional.

17. A growth medium comprising N-cyclopropyl-1-(4-(4-(fluoro-18F)phenyl)pyrimidin-5-yl)-N-methyl piperidine-4-carboxamide or salt thereof.

18. The growth medium of claim 18 comprising a cell.
